SEQUENCE LISTING (2 sequences)

SEQ ID NO: 1 - DNA, 2157 bp, Homo sapiens
  CDS (464)..(1114)
gctcccagccaagaacctcggggccgctgcgcggtggggaggagttccccgaaacccggccgctaagcgaggcctcctcctcccgcagatccgaacggcctgggcggggtcaccccggctgggacaagaagccgccgcctgcctgcccgggcccggggagggggctggggctggggccggaggcggggtgtgagtgggtgtgtgcggggggcggaggcttgatgcaatcccgataagaaatgctcgggtgtcttgggcacctacccgtggggcccgtaaggcgctactatataaggctgccggcccggagccgccgcgccgtcagagcaggagcgctgcgtccaggatctagggccacgaccatcccaacccggcactcacagccccgcagcgcatcccggtcgccgcccagcctcccgcacccccatcgccggagctgcgccgagagccccagggaggtgccatgcggagcgggtgtgtggtggtccacgtatggatcctggccggcctctggctggccgtggccgggcgccccctcgccttctcggacgcggggccccacgtgcactacggctggggcgaccccatccgcctgcggcacctgtacacctccggcccccacgggctctccagctgcttcctgcgcatccgtgccgacggcgtcgtggactgcgcgcggggccagagcgcgcacagtttgctggagatcaaggcagtcgctctgcggaccgtggccatcaagggcgtgcacagcgtgcggtacctctgcatgggcgccgacggcaagatgcaggggctgcttcagtactcggaggaagactgtgctttcgaggaggagatccgcccagatggctacaatgtgtaccgatccgagaagcaccgcctcccggtctccctgagcagtgccaaacagcggcagctgtacaagaacagaggctttcttccactctctcatttcctgcccatgctgcccatggtcccagaggagcctgaggacctcaggggccacttggaatctgacatgttctcttcgcccctggagaccgacagcatggacccatttgggcttgtcaccggactggaggccgtgaggagtcccagctttgagaagtaactgagaccatgcccgggcctcttcactgctgccaggggctgtggtacctgcagcgtgggggacgtgcttctacaagaacagtcctgagtccacgttctgtttagctttaggaagaaacatctagaagttgtacatattcagagttttccattggcagtgccagtttctagccaatagacttgtctgatcataacattgtaagcctgtagcttgcccagctgctgcctgggcccccattctgctccctcgaggttgctggacaagctgctgcactgtctcagttctgcttgaatacctccatcgatggggaactcacttcctttggaaaaattcttatgtcaagctgaaattctctaattttttctcatcacttccccaggagcagccagaagacaggcagtagttttaatttcaggaacaggtgatccactctgtaaaacagcaggtaaatttcactcaaccccatgtgggaattgatctatatctctacttccagggaccatttgcccttcccaaatccctccaggccagaactgactggagcaggcatggcccaccaggcttcaggagtaggggaagcctggagccccactccagccctgggacaacttgagaattccccctgaggccagttctgtcatggatgctgtcctgagaataacttgctgtcccggtgtcacctgcttccatctcccagcccaccagccctctgcccacctcacatgcctccccatggattggggcctcccaggccccccaccttatgtcaacctgcacttcttgttcaaaaatcaggaaaagaaaagatttgaagaccccaagtcttgtcaataacttgctgtgtggaagcagcgggggaagacctagaaccctttccccagcacttggttttccaacatgatatttatgagtaatttattttgatatgtacatctcttattttcttacattatttatgcccccaaattatatttatgtatgtaagtgaggtttgttttgtatattaaaatggagtttgtttgtaaaaaaaaaaaaaaaaaaaa

SEQ ID NO: 2 - protein, 216 aa, Homo sapiens
MRSGCVVVHVWILAGLWLAVAGRPLAFSDAGPHVHYGWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

(19) United States
(12) Patent Application Publication
(10) Pub. No.: US 2005/0107475 A1
(43) Pub. Date: May 19, 2005

(54) Methods of using farnesoid x receptor (frx) agonists

(76) Inventors: Stacey Ann Jones, Durham, NC (US); Steven Anthony Kliewer, Dallas, TX (US); Traci Ann Mansfield, New Haven, CT (US)

Correspondence Address: DAVID J LEVY, CORPORATE INTELLECTUAL PROPERTY;GLAXOSMITHKLINE, FIVE MOORE DR., PO BOX 13398, RESEARCH TRIANGLE PARK, NC, 27709-3398, US

(21) Appl. No.: 10/507,082

(22) PCT Filed: Mar. 19, 2003
(86) PCT No.: PCT/US03/08634

Related U.S. Application Data
(60) Provisional application No. 60/366,463, filed on Mar. 21, 2002.

(51) Int. Cl. A61K 31/47; A61K 31/19

(52) U.S. Cl. 514/571

(57) ABSTRACT

Treatment of human hepatocytes with farnesoid X receptor (FXR) agonists resulted in increased expression of FGF-19. Methods of using FXR agonists to alter cell metabolism, and in pharmaceutical weight loss methods, are described.

### FIELD OF THE INVENTION

The present invention relates to farnesoid X receptor (FXR) agonists and their use in methods of affecting the metabolism of cells, and in pharmaceutical weight loss methods.

### BACKGROUND OF THE INVENTION

Being overweight or obese substantially raises an individual's risk of morbidity from hypertension, dyslipidemia, type 2 diabetes, coronary heart disease, and other conditions. Despite the expected medical benefits, many overweight individuals find it difficult to successfully lose weight by diet management alone. Obesity is recognized as a complex multifactorial condition that develops from the interaction of genetic and environmental factors. See, e.g., Clinical Guidelines on the Identification, Evaluation, and Treatment of Overweight and Obesity in Adults, Am. J. Clin. Nutr. 68:899 (1998).

Various pharmaceutical compounds have been utilized in weight loss treatments. Serotonergic agents that inhibit the reuptake of serotonin are reported to act on the hypothalamus to decrease satiety. However, serious cardiovascular side effects have been reported in some individuals treated with such agents. Fenfluramine and dexfenfluramine, serotonergic agents previously utilized in the United States for the treatment of obesity, have been withdrawn from the U.S. market due to reports of valvular heart disease and primary pulmonary hypertension. (Davidoff et al., Arch Intern Med 161:1429 (2001); Michelakis et al., Am J Med Sci 321:292 (2001); Weissman, Am J Med Sci 321:285 (2001); 2001 PHYSICIANS DESK REFERENCE®, Medical Economics Co., (2000)).

In view of the need for medical weight loss therapies, additional pharmaceutical methods useful in weight control or weight loss are desirable.

### SUMMARY OF THE INVENTION

A first aspect of the present invention is a method of increasing leptin release from the adipocyte cells of a mammalian subject, by administering an FXR agonist to the subject. Leptin release is increased, compared to the leptin release that would occur without FXR agonist administration.

A further aspect of the present invention is a method of decreasing glucose uptake by the adipocyte cells of a mammalian subject, by administering an FXR agonist to the subject. Glucose uptake is decreased compared to that which would occur in the absence of FXR agonist administration.

A further aspect of the present invention is a method of treating a mammalian subject to achieve weight loss, by administration of a pharmaceutically acceptable FXR agonist. The subject's weight is decreased, compared to that which would occur in the absence of FXR agonist treatment.

A further aspect of the present invention is a method of reducing the total body mass of a mammalian subject, by administering a pharmaceutically acceptable FXR agonist. The subject's total body mass is reduced compared to the subject's total body mass that would occur in the absence of FXR agonist treatment.

A further aspect of the present invention is a method of increasing the metabolic rate of a mammalian subject, by administering a pharmaceutically acceptable FXR agonist. The metabolic rate of the subject is increased compared to the rate that would occur in the absence of FXR agonist treatment.

A further aspect of the present invention is a method of increasing serum leptin in a mammalian subject, by administering to a subject a pharmaceutically acceptable FXR agonist. The serum leptin in the subject is thereby increased compared to that would occur in the absence of FXR agonist treatment.

A further aspect of the present invention is a method of inducing expression of FGF19 in a human hepatocyte cell, by administering an FXR agonist to the cell. The cell maybe in vitro.

### DETAILED DESCRIPTION OF THE INVENTION

The present invention relates to the use of Farnesoid X Receptor (FXR) agonists to affect the metabolism of cells, and as a pharmaceutical treatment for weight control and weight loss. The present inventors determined that activation of the nuclear receptor FXR by a bile acid agonist, as well as by a small molecule FXR agonist, caused an increase in transcription of a human Fibroblast Growth Factor gene (hFGF19), leading to an increase in the quantity of mRNA encoding the fibroblast growth factor. Accordingly, in humans expression of FGF19 and its downstream activity can be modulated using FXR agonists. Human FGF19 has been reported to induce leptin release from rat adipocyte cells and decrease glucose uptake by rat adipocyte cells; transgenic mice expressing hFGF19 have been reported to be less fat than their nontransgenic littermates; increased oxygen consumption has been reported in mice administered recombinant FGF-19; and administration of FGF-19 has been suggested as a treatment for obesity (WO 01/18210, Genentech).

FXR

FXR is a member of the nuclear receptor family of ligand-activated transcription factors that includes receptors for the steroid, retinoid, and thyroid hormones (D J. Mangelsdorf, et al., Cell 83:841-850 (1995)). Northern and in situ analysis show that FXR is most abundantly expressed in the liver, intestine, kidney, and adrenal (B M. Forman, et al., Cell 81:687 (1995) and W. Seol, et al., Mol. Endocrinnol. 9:72 (1995)). FXR binds to DNA as a heterodimer with the 9-cis retinoic acid receptor (RXR). The FXR/RXR heterodimer preferentially binds to response elements composed of two nuclear receptor half sites of the consensus AG(G/T)TCA organized as an inverted repeat and separated by a single nucleotide (IR-1 motif) (B M. Forman, et al., Cell 81:687 (1995)). An early report showed that rat FXR is activated by micromolar concentrations of farnesoids such as farnesol and juvenile hormone (B M. Forman, et al., Cell 81:687-693 (1995)). However, these compounds failed to activate the mouse and human FXR, leaving the nature of the endogenous FXR ligand in doubt. Several naturally-occurring bile acids bind to and activate FXR at physiological concentrations (PCT WO 00/37077, published 29 Jun. 2000)). As discussed therein, the bile acids that serve as FXR ligands include chenodeoxycholic acid (CDCA), deoxycholic acid (DCA), lithocholic acid (LCA), and the taurine and glycine conjugates of these bile acids.

Bile acids are cholesterol metabolites that are formed in the liver and secreted into the duodenum of the intestine, where they have roles in the solubilization and absorption of dietary lipids and vitamins. Most bile acids (˜95%) are subsequently reabsorbed in the ileum and returned to the liver via the enterohepatic circulatory system. The conversion of cholesterol to bile acids in the liver is under feedback regulation: bile acids down-regulate the transcription of cytochrome P450 7a (CYP7a), which encodes the enzyme that catalyzes the rate limiting step in bile acid biosynthesis. FXR is involved in both the stimulation and the repression (via CYP7a) of target genes involved in bile acid and cholesterol homeostasis.

FXR Ligands

The bile acids chenodeoxycholic acid (CDCA), deoxycholic acid (DCA), lithocholic acid (LCA), and the taurine and glycine conjugates thereof selectively activate FXR (WO 0037077, Glaxo Group Limited). As used herein, the term “FXR agonist” refers to compounds that achieve at least about 50% activation of FXR relative to CDCA, the appropriate positive control in the assay methods described in PCT Publication No. WO 00/37077 published 29 Jun. 2000 to Glaxo Group Limited, the subject matter of which is incorporated herein by reference in its entirety. Preferably, the FXR agonist compounds used in the methods of this invention achieve at least about 70% activation of FXR in the scintillation proximity assay or the HTRF assay as described in PCT Publication No. WO 00/37077; more preferably, the compounds achieve at least about 80%, 90%, 95%, 97% or greater activation of FXR in the scintillation proximity assay or the HTRF assay as described in PCT Publication No. WO 00/37077.

An FXR agonist for use in the present invention is the compound known as GW4064, as disclosed in PCT Publication No. WO 00/37077 published 29 Jun. 2000 to Glaxo Group Limited, which describes FXR ligand compounds characterized by the following formula (I)

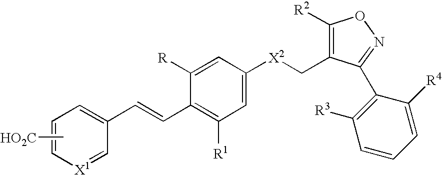

wherein X¹ is CH or N; X² is O or NH; R and R¹ may independently be H, lower alkyl, halogen, or CF₃; R² is lower alkyl; R³ and R⁴ may independently be H, lower alkyl, halogen, CF₃, OH, O-alkyl, or O-polyhaloalkyl.

GW4064, an example of a compound of Formula (I), is a potent and selective FXR ligand and has the following formula (II):

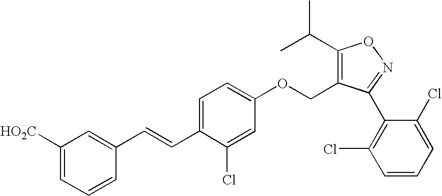

FXR agonists for use in the present invention further include compounds of formula III: 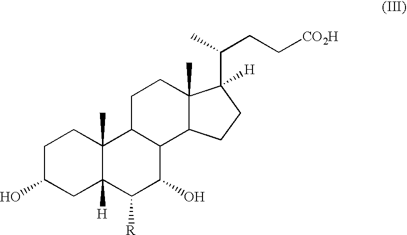
wherein R is ethyl, propyl or allyl, and pharmaceutically acceptable salts, solvates or amino acid conjugates thereof.

Suitable pharmaceutically acceptable salts of the above compounds may be readily determined by one skilled in the art and may include, for example, basic salts such as metallic salts made from aluminium, calcium, lithium, magnesium, potassium, sodium, and zinc or organic salts made from N,N′-dibenzylethylenediamine, chlorprocaine, choline, diethanolamine, ethylendiamine, meglumine (N-methylglucamine), and procaine. Such salts may be prepared using conventional techniques, as are known in the art. As used herein, the tern “solvate” is a crystal form containing the active compound (or a pharmaceutically acceptable salt thereof) and either a stoichiometric or a non-stoichiometric amount of a solvent. Solvents, by way of example, include water, methanol, ethanol, or acetic acid.

As used herein, the term “amino acid conjugates” refers to conjugates of a compound with any suitable amino acid. Preferably, such suitable amino acid conjugates have the added advantage of enhanced integrity in bile or intestinal fluids. Suitable amino acids include but are not limited to glycine and taurine. Thus, the present invention encompasses the use of glycine and taurine conjugates of FXR agonists.

Compounds of formula (III) include compounds selected from the group consisting of 3α,7α-dihydroxy-6α-ethyl-5β-cholan-24-oic acid; 3α,7α-dihydroxy-6α-propyl-5β-cholan-24-oic acid and 3α,7α-dihydroxy-6α-allyl-5β-cholan-24-oic acid and their pharmaceutically acceptable salts, solvates or amino acid conjugates thereof.

The amount of FXR agonist, or pharmaceutically acceptable salt or solvate thereof, which is required to achieve the desired biological effect will depend on a number of factors such as the means of administration, the desired outcome, and the recipient. In general, in treating mammals for weight control or weight loss purposes, a typical daily dose of an FXR agonist of formula (I-III) may be expected to lie in the range of from about 0.01 mg/kg to about 100 mg/kg. This dose may be administered as a single unit dose or as several separate unit doses or as a continuous infusion.

FGF19

FGF19 is a member of the fibroblast growth factor (FGF) family of proteins. Members of this family are known to be involved in tissue repair, angiogenesis, induction of genes containing an FGF-inducible response element (FiRE), mitogenesis, oncogenesis, and differentiation. The biological specificity of FGFs is believed to be partly due to the controlled expression of both the FGFs and the FGF receptors (FGFRs). Four related receptor tyrosine kinases have been identified that bind to members of the FGF family; the presence of heparin or heparan sulfate proteoglycans is believed to be required for the biological activity of FGFs.

The structure and expression of human FGF-19 is described in Nishimura et al., Biochim Biophys Acta 1444:148 (1999). FGF-19 binds with high affinity to the cell surface tyrosine kinase receptor FGF Receptor 4 (FGFR4), and displays selective binding to FGFR4. Xie et al., Cytokine 11:729 (1999).

The sequence for human FGF19 mRNA is provided at GenBank Accession Number AF110400:

| CDS 464-1114 | | |
|---|---|---|
| gctcccagcc aagaacctcg gggccgctgc gcggtgggga ggagttcccc gaaacccggc | 60 | (SEQ ID NO:1) |
| cgctaagcga ggcctcctcc tcccgcagat ccgaacggcc tgggcggggt caccccggct | 120 | |
| gggacaagaa gccgccgcct gcctgcccgg gcccggggag ggggctgggg ctggggccgg | 180 | |
| aggcggggtg tgagtgggtg tgtgcggggg gcggaggctt gatgcaatcc cgataagaaa | 240 | |
| tgctcgggtg tcttgggcac ctacccgtgg ggcccgtaag gcgctactat ataaggctgc | 300 | |
| cggcccggag ccgccgcgcc gtcagagcag gagcgctgcg tccaggatct agggccacga | 360 | |
| ccatcccaac ccggcactca cagccccgca gcgcatcccg gtcgccgccc agcctcccgc | 420 | |
| acccccatcg ccggagctgc gccgagagcc ccagggaggt gccatgcgga gcgggtgtgt | 480 | |
| ggtggtccac gtatggatcc tggccggcct ctggctggcc gtggccgggc gccccctcgc | 540 | |
| cttctcggac gcggggcccc acgtgcacta cggctggggc gaccccatcc gcctgcggca | 600 | |
| cctgtacacc tccggccccc acgggctctc cagctgcttc ctgcgcatcc gtgccgacgg | 660 | |
| cgtcgtggac tgcgcgcggg gccagagcgc gcacagtttg ctggagatca aggcagtcgc | 720 | |
| tctgcggacc gtggccatca agggcgtgca cagcgtgcgg tacctctgca tgggcgccga | 780 | |
| cggcaagatg caggggctgc ttcagtactc ggaggaagac tgtgctttcg aggaggagat | 840 | |
| ccgcccagat ggctacaatg tgtaccgatc cgagaagcac cgcctcccgg tctccctgag | 900 | |
| cagtgccaaa cagcggcagc tgtacaagaa cagaggcttt cttccactct ctcatttcct | 960 | |
| gcccatgctg cccatggtcc cagaggagcc tgaggacctc aggggccact tggaatctga | 1020 | |
| catgttctct tcgcccctgg agaccgacag catggaccca tttgggcttg tcaccggact | 1080 | |
| ggaggccgtg aggagtccca gctttgagaa gtaactgaga ccatgcccgg gcctcttcac | 1140 | |
| tgctgccagg ggctgtggta cctgcagcgt gggggacgtg cttctacaag aacagtcctg | 1200 | |
| agtccacgtt ctgtttagct ttaggaagaa acatctagaa gttgtacata ttcagagttt | 1260 | |
| tccattggca gtgccagttt ctagccaata gacttgtctg atcataacat tgtaagcctg | 1320 | |
| tagcttgccc agctgctgcc tgggccccca ttctgctccc tcgaggttgc tggacaagct | 1380 | |
| gctgcactgt ctcagttctg cttgaatacc tccatcgatg gggaactcac ttcctttgga | 1440 | |
| aaaattctta tgtcaagctg aaattctcta attttttctc atcacttccc caggagcagc | 1500 | |
| cagaagacag gcagtagttt taatttcagg aacaggtgat ccactctgta aaacagcagg | 1560 | |
| taaatttcac tcaaccccat gtgggaattg atctatatct ctacttccag ggaccatttg | 1620 | |
| cccttcccaa atccctccag gccagaactg actggagcag gcatggccca ccaggcttca | 1680 | |
| ggagtagggg aagcctggag ccccactcca gccctgggac aacttgagaa ttccccctga | 1740 | |
| ggccagttct gtcatggatg ctgtcctgag aataacttgc tgtcccggtg tcacctgctt | 1800 | |
| ccatctccca gcccaccagc cctctgccca cctcacatgc ctccccatgg attggggcct | 1860 | |
| cccaggcccc ccaccttatg tcaacctgca cttcttgttc aaaaatcagg aaaagaaaag | 1920 | |
| atttgaagac cccaagtctt gtcaataact tgctgtgtgg aagcagcggg ggaagaccta | 1980 | |
| gaaccctttc cccagcactt ggttttccaa catgatattt atgagtaatt tattttgata | 2040 | |
| tgtacatctc ttattttctt acattattta tgcccccaaa ttatatttat gtatgtaagt | 2100 | |
| gaggtttgtt ttgtatatta aaatggagtt tgtttgtaaa aaaaaaaaaa aaaaaaa | 2157 | |

Human FGF19 polypeptides and nucleic acid molecules encoding the same are described in WO 0118210 (Genentech, Inc.), EP1032668 (Genentech), and WO 0118209 (Curagen Corporation). The production of transgenic mice expressing human FGF19 (using the promoter for myosin light chain to result in muscle specific transcription of the transgene) is reported in WO 0118210, where it is further reported that these mice demonstrated increased food intake and increased metabolic rate (evidenced by their rate of oxygen consumption and increased urine output). However, the transgenic mice weighed significantly less than their non-transgenic littermates, despite their increased food intake. The transgenic mice had normal linear growth, and normal body temperature and bone length. It is postulated in WO0118210 that the decreased body weight of transgenic hFGF19 mice is due to decreased adiposity; leptin (which is reported to correlate closely with adipose tissue mass in humans and rodents) is decreased in the transgenic mice. Infusion of FGF19 to non-transgenic mice was reported to cause an increase in food intake. It is stated in WO0118210 that FGF19 decreases adiposity without altering muscle mass or long bone formation, and that FGF19 is indicated as a therapeutic in the treatment of obesity and related conditions. Further, the effects of a high-fat diet on glucose tolerance in transgenic mice expressing hFGF19 were compared to the effects in non-transgenic littermates. Transgenic rice fed a high-fat diet for ten weeks were subjected to a glucose tolerance test; the majority of the non-transgenic mice fed a high-fat diet were defined as diabetic, whereas none of the transgenic mice fed a comparable high-fat diet were defined as diabetic by the glucose tolerance test. WO 01/18210.

Definitions

“Mammal” as used herein includes primates and humans, as well as livestock and companion animals.

“Pharmaceutical weight loss treatment” as used herein refers to administration of a pharmaceutical compound to a subject whose weight is greater than a medically acceptable or medically desirable amount, to achieve a reduction in the subject's weight. “pharmaceutical treatment of obesity” is an aspect of pharmaceutical weight loss treatment and refers to such treatment for individuals whose body mass meets an accepted medical definition of obesity. One commonly accepted measure of overweight in humans is the Body Mass Index (BMI); overweight may be defined as a BMI of at least 25 kg/m², with obesity defined as a BMI of at least 30 kg/m². Pharmaceutical weight loss treatment may be accompanied by a change in diet and/or other behavioral modifications such as support groups and/or patient education. As used herein, pharmaceutical weight loss treatment does not imply a “cure” for obesity or permanent weight loss. “Pharmaceutical weight maintenance treatment” as used herein refers to administration of a pharmaceutical compound to a subject as an aid in maintaining a desired weight. As described herein, FXR agonists are useful in such treatments described above.

Body Mass Index is a numerical measurement of relative weight for height, and has been significantly correlated with total body fat content. BMI is calculated as weight (kg)/height squared (m²). See, e.g., Clinical Guidelines on the Identification, Evaluation, and Treatment of Overweight and Obesity in Adults. Am. J. Clin. Nutr. 68:899 (1998).

As used herein, an FXR ligand pharmaceutical compound for the treatment of obesity or for weight loss treatment is one in which administration (in an appropriate pharmaceutical formulation and in a therapeutically effective amount) to a mammal, and preferably to humans, has been shown to increase weight loss over time, compared to the change in weight that would have occurred had the subject not been administered the compound. Therapeutically effective amounts of such compounds for use in treatment of obesity or for weight loss can be readily determined by those skilled in the art using, e.g., dose-response studies.

Treatment of a subject with an FXR agonist pharmaceutical compound comprises administration of an effective amount (for the condition being treated) of the pharmaceutical agent to a subject. The dose of agent is determined according to methods known and accepted in the pharmaceutical arts, and can be determined by those skilled in the art.

Differential Gene Expression

The present study utilized differential gene expression analysis as performed by CuraGen Corporation (New Haven, Conn.) using transcript profiling technology. See Nat Biotechnol 17:798-803, 1999; see also U.S. Pat. Nos. 5,871,697 and 5,972,693. In brief, RNA is extracted from samples and cDNA synthesis is carried out. The cDNAs are then digested with multiple pairs of different restriction enzymes (standard set of 96 different pairs of restriction enzymes with 6 base-pair recognition sites (subsequence pairs)), and the digested products are ligated to complementary adapters containing standardized PCR priming sequences. Multiple PCR cycles are carried out using one biotinylated adapter-specific primer and one fluorescently labeled adapter-specific primer. Following PCR amplification, the biotin-labeled DNA is purified on immobilized streptavidin. Denatured single stranded cDNA fragments are then sized using capillary electrophoresis, and the fluorescently labeled fragments are detected by laser excitation. Since the biotin label is needed for purification and the fluorescent label is needed for detection, all analyzed fragments result from restriction digestion with both enzymes.

Differentially expressed peaks are identified by comparing the composite traces of the experimental samples vs. the control samples using bioinformatics algorithms. Each differentially expressed peak is then compared to a database containing a ‘virtual’ restriction digest of a human sequence database. (The database was constructed by performing in silico digests on a human sequence database, using all subsequence pairs as described above; for each transcribed DNA sequence, the computed fragment sizes and associated restriction enzymes (bands) are stored in the database.) By comparing the differentially expressed peaks with the database, genes are identified that are predicted to generate DNA fragments with lengths and terminal sequences that match the restriction enzymes used and the detected length of the differentially expressed peaks. A single differentially expressed band may be part of one or more genes. Additional bands from at least one of these genes should be present and should also be differentially expressed.

Thus, confirmation and gene identification of differentially expressed bands can occur by two routes. One method is GeneCalling/Poisoning. In this case, cDNA fragments representing differentially expressed genes can be identified by database searching with the 6 base-pair restriction enzyme recognition sequences at the fragment ends and the exact length of each fragment (determined electrophoretically, subtracting linker length). Database searching for genes predicted to have restriction fragments of matching lengths enables the identification of such genes whose sequences reside in that database (a “GeneCall”). The detection of multiple fragments derived from the same gene which show differential expression of the same directional modulation increases the likelihood that the prediction of the gene identity is correct. The differentially expressed gene fragment and the predicted gene sequence identified by the database lookup, can then be linked through a positive poisoning reaction. In this process, the reaction containing the fragment of interest is performed a second time using the same end primers, but in the presence or absence of an excess of an unlabeled oligonucleotide whose sequence is derived from the predicted gene fragment. If the identity of the fragment was predicted correctly, the unlabeled oligonucleotide will out-compete the universal oligonucleotide for priming that fragment and, in the resulting chromatogram, will appear to ablate that peak specifically without affecting the amplification of the other fragments.

An alternative method for gene identification and confirmation, Isolation/Poisoning, relies on the isolation of the differentially expressed fragment from the re-amplified GeneCalling chemistry reaction from a preparative gel. The gel-purified fragment is re-amplified and cloned in a standard PCR product cloning vector. The insert is sized and sequenced, and primers for poisoning are designed from one or both ends of the cloned fragment. The poisoning reaction is performed and analyzed as described above. Successful ablation of the peak using the unlabeled oligonucleotide based on the cloned sequence identifies the sequence as corresponding to the original differentially expressed gene fragment. Subsequently, the gene identification is obtained by standard BLASTN or BLASTX analysis of the poisoning cloned sequence.

The present invention provides methods of using FXR agonists to modulate FGF-19 expression by mammalian cells and, in mammalian organisms, to thereby modulate the downstream effects of FGF-19. The present methods further provide methods of using FXR agonists to modulate the metabolism of mammals , and as a treatment for weight loss or weight control in mammals. Such methods include using FXR agonist administration to a mammal to achieve an increase in circulating or serum leptin levels. The increase in leptin levels is compared to that which would otherwise occur (i.e., occur in the absence of the FXR agonist administration).

While not wishing to be held to a single theory, the present inventors believe that administration of an FXR agonist results in increased expression of genes that play a role in metabolism and weight maintenance. FXR agonists are shown herein to increase expression of FGF19 in human cells (hepatocytes). A protein homologous to FGF19, or having high sequence similarity to FGF19, may not naturally occur in all mammals. However it is postulated that non-human mammals possess fibroblast growth factors (or biomolecules having similar functional effects) that provide an FXR-mediated pathway similar to the FXR-FGF19 pathway, such that administration of FXR agonists to the animal will affect adipocyte function, metabolism and/or overall weight maintenance (or weight loss) in the manner described herein.

Leptin is a hydrophilic protein secreted from white adipocytes. Administration of recombinant leptin to ob/ob mice, which lack the functional protein and are obese, results in a reduction of food intake and an increase in energy expenditure. Halas et al., Science 269: 540-549 (1995). Insulin, glucocorticoids, TNFα, and interleukin-1 have been reported to stimulate the expression of the ob gene; fasting and the administration of isoprenaline or selective β3-adrenoceptor agonists have been reported to cause a decrease in ob gene expression and a corresponding decrease in circulating leptin. Serum concentrations of leptin have been reported as reflecting the nutritional status and body fat mass of individuals.

The present invention further provides a method of decreasing glucose uptake by the cells of a mammal (particularly adipocytes or white adipocyte cells) by administering an FXR agonist to the mammal in an amount effective to decrease glucose uptake by such cells. The decrease in glucose uptake is relative to that which would occur in the absence of the FXR agonist treatment.

The present invention further provides a method of treating a mammalian subject in need of weight loss or weight maintenance treatment, by administering to the subject a pharmaceutically acceptable FXR agonist in an amount effective to decrease said subject's weight. The decrease in weight is relative to the change in weight which would occur (or would be expected to occur) in the absence of FXR agonist treatment. The present methods include pharmaceutical weight loss treatment of humans.

The present uses of FXR agonists include methods of increasing the metabolic rate of a mammalian subject, or increasing serum (or circulating) leptin in a mammalian subject, comprising administering to a subject a pharmaceutically acceptable FXR agonist in an amount effective to increase the metabolic rate or increase the serum leptin concentrations of the subject. The increase in metabolic rate may be assessed by any suitable means as is known in the art, such as by measuring oxygen consumption and/or urine output in a controlled setting. The increase in metabolic rate or leptin concentration is relative to that would occur in the absence of FXR agonist treatment.

While not wishing to be held to a single theory underlying the present invention, the present inventors believe that, in a mammal in which both Farnesoid X Receptors and FGF19 (or a protein having similar function to FGF19) are naturally present, administration of an FXR agonist to the animal will increase levels of the fibroblast growth factor (or similar protein), will affect adipocytes (including but not limited to increased release of leptin and/or decreased uptake of glucose), and will affect the animal's overall metabolic and/or weight status.

Also provided herein is a method of inducing FGF19 expression from human cells, preferably human hepatocytes, by administering an FXR agonist to said cell or exposing said cell to an FXR agonist; the cells may be in vitro. FGF19 protein secreted into cell culture media may be isolated and purified using any suitable technique as is known in the art. All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.

### EXAMPLES

### Example 1

### Differential Expression of FGF19 in Human Hepatocytes Treated with FXR Agonists

The pattern of gene expression induced in human hepatocytes after treatment with an FXR ligand was measured.

Materials and Methods

Nine samples of human hepatocytes were used: Human hepatocytes treated with DMSO control (3 samples), human hepatocytes treated with chenodeoxycholic acid (CDCA; 3 samples), and human hepatocytes treated with FXR agonist GW4064X (3 samples). RNA was extracted from the cells and gene expression analysis was performed by CuraGen Corporation (New Haven, Conn.) using transcript profiling technology as described above. See Nat Biotechnol 17:798-803, 1999; see also U.S. Pat. Nos. 5,871,697 and 5,972,693.

Three independent reactions from each cDNA sample were compared for quality of electrophoretic peak resolution and reproducibility of peak patterns. Composite traces from each sample were generated, and then compared among the three independent samples for peak quality and reproducibility. The resulting traces represent the total gene expression profile for the tissue sampled and treatment. The databases for each sample were compared to identify differences in gene expression resulting from the different treatments. The composite traces calculated for each sample group, based on average peak height and variance, were compared among sample groups using software designed to identify peaks representing differential expression.

Bands (representing gene fragments) that did not change expression levels in either of the treatments (compared to control group) were filtered out. Bands showing differential expression in at least one of the treatments by a factor greater than or equal to +/−1.5 fold (compared to control) were included in the analysis.

Results

Expression of human Fibroblast Growth Factor 19 (hFGF19) was increased 36.4-fold in human hepatocytes treated with FXR agonist compound GW4064X compared to control cells; expression of hFGF19 was increased 5.6-fold in human hepatocytes treated with CDCA compared to control cells. These results indicated that treatment of a cell with an FXR agonist increases transcription of hFGF19.

### Example 2

### Effect of FXR Agonist on Leptin Release from Adipocytes In Vitro

The addition of recombinant human FGF-19 to cultures of primary rat adipocytes is reported to increase the release of leptin from the cells (WO 0118210). As noted in Example 1, herein, expression of human Fibroblast Growth Factor 19 (hFGF19) was increased in human hepatocytes treated with FXR agonist compounds, compared to control cells.

The present study investigates the use of an FXR agonist to induce expression of FGF-19 in liver cells. Increased secretion of the hFGF19 protein can thereby increase the release of leptin from adipocytes.

Cultures of rat adipocytes are established using any suitable means as is known in the art. One suitable method harvests fibroblastic preadipocytes from the inguinal fat deposit of sucking rats, which are then cultured and induced to differentiate into mature adipocytes. Following differentiation, the ob (leptin) gene is expressed and leptin is secreted into the culture medium (typically by day 4 after induction). Mitchell et al., Biochem Biophys Res Comm, 230:360 (1997). Alternatively, isolated primary adipocytes may be cultured (see, e.g., Hardie et al., Horm Metab Res 28:685 (1996)), or excised pieces of adipose tissue may be maintained in cell culture conditions (see, e.g., Ott et al., Exp. Biol. Med. 226(9):841 (2002)).

An FXR agonist is added to liver cells cultures (primary human hepatocytes or HuH7 cells) in order to induce expression and secretion of FGF19. Suitable FXR agonists include compounds of Formulas I-III as described herein. The resulting conditioned media containing FGF19 is then added to adipocyte cell cultures in a range of concentrations. Control cell cultures are also prepared (without FXR agonist), to provide control media which is added to control cultures of adipocyte cells.

Leptin secreted into culture medium from the adipocyte cell cultures is measured by any suitable means as is known in the art, including e.g., Enzyme Linked Immunosorbent Assay (ELISA) or Radio-Immunoassay (RIA) (Rat Leptin RIA kit, Linco Research Inc., St. Charles, Mo.). Leptin secretion is measured as a function of FXR agonist concentration, (and/or conditioned media concentration, and/or FGF19 concentration) and time elapsed after addition of conditioned media to adipocyte cell cultures, and is compared to control cell cultures.

### Example 3

### Effect of FXR Agonist on Glucose Uptake by Adipocytes In Vitro

The addition of recombinant human FGF-19 to cultures of primary rat adipocytes has been reported to decrease the uptake of glucose (WO 0118210). The present study investigates the use of an FXR agonist to induce expression of FGF-19 in liver cells, and the effect of hFGF19 protein on the uptake of glucose by rat white adipocytes.

Cultures of primary rat adipocytes and human liver cells are established using any suitable means as is known in the art, as discussed above.

An FXR agonist is added to liver cell cultures (primary human hepatocytes or HuH7 cells) to induce expression and secretion of FGF19. Control liver cell cultures (without exposure to FXR agonist) are also prepared. Suitable FXR agonists include compounds of Formulas I-III as described herein. Conditioned media obtained from the liver cell cultures exposed to FXR agonist (and control medium) are then added to adipocyte cell cultures in a range of concentrations. Glucose uptake by adipocytes is measured by any suitable means as is known in the art, as a function of FXR agonist concentration (and/or conditioned media concentration, and/or FGF19 concentration) and time elapsed after addition of FXR agonist, and is compared to control cell cultures.

### Example 4

### FXR Agonist in Mice

Infusion of FvB mice with recombinant human FGF-19 (1 mg/kg/day, delivered intravenously by an osmotically driven implanted pump) has been reported to result in increased food intake and increased oxygen consumption compared to mice infused with carrier alone (WO 01 18210, Genentech).

Non-transgenic FvB mice are administered an FXR agonist in a suitable carrier; control mice are administered the carrier alone. Suitable FXR agonists include compounds of Formulas I-III as described herein. Administration may be by any means suitable for the particular FXR agonist (e.g., oral, subcutaneous, intravenous, or via implanted pump). Mice receiving FXR agonist may be further divided into groups receiving varying dosage regimes of FXR agonist. Treated and control mice may be further divided according to feeding regime (e.g., ad libitum normal diet; controlled portion normal diet; ad libitum high fat diet; controlled portion high fat diet).

Weight, and optionally oxygen consumption, of the treated mice and the controls are measured at various time points and compared. Weight may be measured by any suitable means, including total animal weight and/or sacrifice of the animals after a set amount of time and measurement of the weight of specific fat deposits (e.g., epididymal, retroperitoneal with peri-renal). Circulating leptin may also be measured at various time points and compared among treatment group(s) and controls. A decrease in weight, or decreased rate of weight gain, and/or decrease in adiposity (as indicated by circulating leptin levels), in treated mice relative to controls indicates that the FXR agonist decreases adiposity, and indicates FXR agonists as a therapeutic in the treatment of obesity in mammals.

### Example 5

### FXR Agonist in Rats

A strain of rat suitable for use in laboratory experiments and not known to have metabolic defects are administered an FXR agonist in a suitable carrier and at a range of FXR dosages; control rats are administered the carrier alone. Suitable FXR agonists include compounds of Formulas I-III as described herein. Administration may be by any means suitable for the particular FXR agonist (e.g., oral, subcutaneous, intravenous, or via implanted pump). Treated and control rats may be further divided according to feeding regime (e.g., ad libitum normal diet; controlled portion normal diet; ad libitum high fat diet; controlled portion high fat diet).

Weight, and optionally oxygen consumption, of the treated animals and the controls are measured at various time points and compared. Weight may be measured by any suitable means, including total animal weight and/or sacrifice of the animals after a set amount of time and measurement of the weight of specific fat deposits (e.g., epididymal, retroperitoneal with peri-renal). Circulating leptin may also be measured at various time points and compared among treatment group(s) and controls. A decrease in weight, or decreased rate of weight gain, and/or decrease in adiposity (as indicated by circulating leptin levels), in treated animals relative to controls indicates that the FXR agonist decreases adiposity, and indicates FXR agonists as a therapeutic in the treatment of obesity in mammals.

### Example 6

### FXR Agonist in Primates

A group of non-human primates suitable for use in laboratory experiments and not known to have any metabolic defects are administered an FXR agonist in a suitable carrier and at a range of FXR dosages; control primates are administered the carrier alone. Suitable FXR agonists include compounds of Formulas I-III as described herein. Administration may be by any means suitable for the particular FXR agonist (e.g., oral, subcutaneous, intravenous, or via implanted pump). Treated and control animals may be further divided according to feeding regime (e.g., ad libitum normal diet; controlled portion normal diet; ad libitum high fat diet; controlled portion high fat diet).

Weight, and optionally oxygen consumption, of the treated animals and the controls are measured at various time points and compared. Weight may be measured by any suitable means, including total animal weight and/or sacrifice of the animals after a set amount of time and measurement of the weight of specific fat deposits (e.g., epididymal, retroperitoneal with peri-renal). Circulating leptin or expression of FGF19 may also be measured at various time points and comparedamong treatment group(s) and controls. A decrease in weight, or decreased rate of weight gain, and/or decrease in adiposity (as indicated by circulating leptin levels), in treated animals relative to controls indicates that the FXR agonist decreases adiposity, and indicates FXR agonists as a therapeutic in the treatment of obesity in mammals.

1. A method of increasing leptin release from adipocyte cells of a mammalian subject, comprising administering an FXR agonist to said subject in an amount effective to increase leptin release, compared to that which would occur in the absence of said FXR agonist administration.

2. A method of decreasing glucose uptake by adipocyte cells of a mammalian subject, comprising administering an FXR agonist to said subject in an amount effective to decrease glucose uptake compared to that which would occur in the absence of said FXR agonist administration.

3. A method of treating a mammalian subject in need of weight loss treatment, comprising administering to said subject a pharmaceutically acceptable FXR agonist in an amount effective to decrease said subject's weight, compared to the weight loss that would occur in the absence of FXR agonist treatment.

4. A method of reducing total body mass of a mammalian subject, comprising administering to a subject a pharmaceutically acceptable FXR agonist in an amount effective to reduce said subject's total body mass, compared to the subject's total body mass that would occur in the absence of FXR agonist treatment.

5. A method of increasing the metabolic rate of a mammalian subject, comprising administering to a subject a pharmaceutically acceptable FXR agonist in an amount effective to increase the metabolic rate of said subject, compared to the rate that would occur in the absence of FXR agonist treatment.

6. A method of increasing serum leptin in a mammalian subject, comprising administering to a subject a pharmaceutically acceptable FXR agonist in an amount effective to increase serum leptin in said subject, compared to that would occur in the absence of FXR agonist treatment.

7. A method according to claim 1, wherein said FXR agonist is selected from the group consisting of GW4064; 3α,7α-dihydroxy-6α-ethyl-5β-cholan-24-oic acid; 3α,7α-dihydroxy-6α-propyl-5β-cholan-24-oic acid; and 3α,7α-dihydroxy-6α-allyl-5β-cholan-24-oic acid, and pharmaceutically acceptable salts, solvates or amino acid conjugates thereof.

8. (Canceled)

9. (Canceled)

10. (Canceled)

11. A method of inducing expression of FGF19 in a human hepatocyte cell, comprising administering an FXR agonist to said cell.

12. A method according to claim 11, wherein said FXR agonist is selected from the group consisting of GW4064; 3α,7α-dihydroxy-6α-ethyl-5β-cholan-24-oic acid; 3α,7α-dihydroxy-6α-propyl-5β-cholan-24-oic acid; and 3α,7α-dihydroxy-6α-allyl-5β-cholan-24-oic acid, and pharmaceutically acceptable salts, solvates or amino acid conjugates thereof.

13. A method according to claim 11 where said cell is in vitro.

14. A method according to claim 2, wherein said FXR agonist is selected from the group consisting of GW4064; 3α,7α-dihydroxy-6α-ethyl-5β-cholan-24-oic acid; 3α,7α-dihydroxy-6α-propyl-5β-cholan-24-oic acid; and 3α,7α-dihydroxy-6α-allyl-5β-cholan-24-oic acid, and pharmaceutically acceptable salts, solvates or amino acid conjugates thereof.

15. A method according to claim 3, wherein said FXR agonist is selected from the group consisting of GW4064; 3α,7α-dihydroxy-6α-ethyl-5β-cholan-24-oic acid; 3α,7α-dihydroxy-6α-propyl-5β-cholan-24-oic acid; and 3α,7α-dihydroxy-6α-allyl-5β-cholan-24-oic acid, and pharmaceutically acceptable salts, solvates or amino acid conjugates thereof.

16. A method according to claim 3, wherein said FXR agonist is selected from the group consisting of GW4064; 3α,7α-dihydroxy-6α-ethyl-5β-cholan-24-oic acid; 3α,7α-dihydroxy-6α-propyl-5β-cholan-24-oic acid; and 3α,7α-dihydroxy-6α-allyl-5β-cholan-24-oic acid, and pharmaceutically acceptable salts, solvates or amino acid conjugates thereof.

17. A method according to claim 4, wherein said FXR agonist is selected from the group consisting of GW4064; 3α,7α-dihydroxy-6α-ethyl-5β-cholan-24-oic acid; 3α,7α-dihydroxy-6α-propyl-5β-cholan-24-oic acid; and 3α,7α-dihydroxy-6α-allyl-5β-cholan-24-oic acid, and pharmaceutically acceptable salts, solvates or amino acid conjugates thereof.

18. A method according to claim 5, wherein said FXR agonist is selected from the group consisting of GW4064; 3α,7α-dihydroxy-6α-ethyl-5β-cholan-24-oic acid; 3α,7α-dihydroxy-6α-propyl-5β-cholan-24-oic acid; and 3α,7α-dihydroxy-6α-allyl-5β-cholan-24-oic acid, and pharmaceutically acceptable salts, solvates or amino acid conjugates thereof.

19. A method according to claim 6, wherein said FXR agonist is selected from the group consisting of GW4064; 3α,7α-dihydroxy-6α-ethyl-5β-cholan-24-oic acid; 3α,7α-dihydroxy-6α-propyl-5β-cholan-24-oic acid; and 3α,7α-dihydroxy-6α-allyl-5β-cholan-24-oic acid, and pharmaceutically acceptable salts, solvates or amino acid conjugates thereof.
